(19) United States
(12) Patent Application Publication
(10) Pub. No.: US 2023/0129819 A1
(43) Pub. Date: Apr. 27, 2023

(54) PREPARATION METHOD FOR ANIMAL MODEL OF ALZHEIMER'S DISEASE AND ANIMAL MODEL OF ALZHEIMER'S DISEASE PREPARED BY THE SAME

(71) Applicant: KOREA INSTITUTE OF SCIENCE AND TECHNOLOGY, Seoul (KR)

(72) Inventors: Soo-Jin OH, Seoul (KR); Sunhwa LIM, Seoul (KR); Min-Ho NAM, Seoul (KR); Ki Duk PARK, Seoul (KR); Seung Eun LEE, Seoul (KR)

(73) Assignee: KOREA INSTITUTE OF SCIENCE AND TECHNOLOGY, Seoul (KR)

(21) Appl. No.: 17/972,875

(22) Filed: Oct. 25, 2022

(30) Foreign Application Priority Data
Oct. 25, 2021 (KR) 10-2021-0142927

(51) Int. Cl. C12N 15/85; A61K 49/00; A01K 67/027

(52) U.S. Cl. CPC C12N 15/8509; A61K 49/0008; A01K 67/0278; C12N 2015/8545; C12N 2750/14143; C12N 2710/10071; A01K 2207/15; A01K 2217/052; A01K 2227/105; A01K 2267/0312

(57) ABSTRACT

The present invention relates to a preparation method for an animal model with Alzheimer's disease by injecting a human mutant tau (AAV-hTau) vector and adenovirus into an animal. The preparation method for an AD animal model provided by the present invention may contribute to the development of the field of treatment technology for treating AD since the preparation method causes AD pathology to appear as early as 8 months old and facilitates studies on AD target treatment strategies and tau pathology.

### TECHNICAL FIELD

The present invention relates to a preparation method for an animal model of Alzheimer's disease and to an animal model of Alzheimer's disease prepared by the same.

### BACKGROUND ART

The increase in the number of dementia patients worldwide due to the phenomenon of super-aging is currently emerging as a serious social problem. According to the National Assembly Budget Office in 2014, the prevalence of dementia in those over the age of 65 is expected to increase to 840,000 in 2020 and 2.17 million in 2050, and thus the scale of social costs due to dementia is also expected to increase rapidly from 11.7 trillion won in 2013 to 23.1 trillion won in 2030, 34.2 trillion won in 2040, and 43.2 trillion won in 2050.

Alzheimer's disease (AD), which is the most common degenerative brain disease that causes dementia, and which has a gradual onset and progressively deteriorates cognitive functions including memory, has features such as neurofibrillary tangles (NFTs) that are composed of extracellular beta-amyloid (Aβ) plaques and hyperphosphorylated tau protein and accumulate within cells (Crowther RA (1991) Straight and paired helical filaments in Alzheimer disease have a common structural unit. Proc Natl Acad Sci USA 88:2288-92).

In understanding the mechanisms of Alzheimer's disease (AD) and establishing novel therapeutic strategies, the development of ideal animal models that can mimic human progressive neuropathology and cognitive impairment is important.

To elucidate the precise pathophysiology of AD and to develop and evaluate effective therapeutic strategies, several AD mouse models, including 3×Tg, 5×fad, and amyloid precursor protein/presenilin 1 (APP/PS1), have been utilized. The APP/PS1 mouse model exhibits plaque pathology exacerbated by overproduction of Aβ in the early stages of AD and has been widely used in AD neuropathology and therapeutic studies.

However, neuron loss and NFT pathology have not been observed in aged APP/PS1 mice even in the presence of Aβ. In addition, in order to exhibit the above-mentioned neurodegeneration, cognitive impairment, and neuropathological changes in vivo, a time of one year or more is required, but it is greatly expensive to maintain mice for one year or more, and there is a problem in that AD studies are delayed since features such as neuron loss and neurofibrillary tangles do not appear, and thus the memory deficit is not clearly observed.

With this background, the present inventors have induced overexpression of human mutant tau protein and astrogliosis in APP/PS1 mice, thereby completing a mouse model that exhibits Aβ pathology and acceleration of astrogliosis as well as NFT pathology and neuron loss, which have not previously appeared in 8-month-old mice, and can reflect the pathological features of AD in humans.

### DISCLOSURE

### Technical Problem

An object of the present invention is to provide a preparation method for an animal model of Alzheimer's disease (AD), comprising injecting 1) a human mutant tau protein expression vector; and 2) adenovirus into an animal other than a human.

Another object of the present invention is to provide an animal model prepared by way of the preparation method.

Still another object of the present invention is to provide a screening method for a prophylactic or therapeutic agent for Alzheimer's disease, comprising (1) administering a test agent to an AD animal model; and 2) comparing the animal model of step (1) with a control animal model and selecting the test agent as a prophylactic or therapeutic agent for Alzheimer's disease when symptoms of Alzheimer's disease are improved.

### Technical Solution

This will be described in detail as follows. Each description and embodiment disclosed in the present invention may also be applied to other descriptions and embodiments. That is, all combinations of various elements disclosed in the present invention fall within the scope of the present invention. Further, the scope of the present invention is not limited by the specific description below.

Further, those skilled in the art will recognize or be able to ascertain, using no more than routine experimentation, many equivalents to the specific embodiments of the present invention described herein. Further, these equivalents should be interpreted to fall within the scope of the present invention.

As an aspect for achieving the objects, the present invention provides a preparation method for an animal model of Alzheimer's disease (AD), comprising injecting 1) a human mutant tau protein (AAV-hTau) vector; and 2) adenovirus into an animal other than a human.

As used herein, the term “tau protein” refers to a group of six highly soluble isoform proteins produced by alternative splicing in the microtubule-associated protein tau (MAPT) gene. The tau protein mainly serves to maintain the stability of microtubules in axons, and is abundant in neurons of the central nervous system (CNS). Although not common elsewhere, the tau protein is expressed at very low levels in CNS astrocytes and oligodendrocytes. Pathologies of the nervous system, such as AD and Parkinson's disease, and dementia are associated with the tau protein, which has become hyperphosphorylated insoluble aggregates called NFTs. The origin of the tau protein of the present invention is not limited, but the tau protein may be specifically derived from humans, monkeys, pigs, cattle, horses, sheep, dogs, rabbits, mice, and the like and more specifically may be derived from humans.

The sequence of the human mutant tau protein of the present invention can be obtained from the GenBank of NCBI. The human mutant tau protein of the present invention may have a P301 L mutation.

As used herein, the “P301L” is linked to chromosome 17 (FTDP-17), and is a tau mutation most commonly observed in patients with frontotemporal dementia accompanied by Parkinson's disease.

As used herein, the term “adenovirus” refers to a medium-sized (90 nm to 100 nm), non-enveloped virus. In the present invention, intracerebral injection of adenovirus may cause neuroinflammation including reactive astrogliosis. In particular, adenovirus can exacerbate AD pathology by causing astrogliosis and/or neurodegeneration. The adenovirus of 2) of the present invention may include a promoter and a reporter protein which are specifically expressed in astrocytes so as to confirm the presence of adenovirus infection or astrogliosis. For example, the adenovirus may include a GFAP promoter. The adenovirus may include a reporter protein fermented by a GFAP promoter. In an embodiment, the reporter protein may be a fluorescent protein.

The adenovirus may include a GFAP promoter and/or a fluorescent protein.

As used herein, the “glial fibrillary acidic protein (GFAP)” is a protein encoded by the GFAP gene. The GFAP is an intermediate filament (IF) protein of type III that is expressed by numerous cell types of the CNS including astrocytes and ependymal cells during development. The “GFAP promoter” of the present invention refers to an expression control sequence that controls the expression of GFAP gene. In the present invention, the GFAP promoter may be one for confirming the presence of adenovirus infection or presence of astrogliosis induction. In order to confirm the presence of adenovirus infection, presence of astrogliosis induction, or the operation of the GFAP promoter, the adenovirus may include an appropriate reporter protein in a downstream region of the GFAP promoter.

Since the GFAP promoter is specifically expressed in astrocytes throughout the CNS, once astrogliosis is induced by the introduction of adenovirus, the GFAP promoter will be activated in astrocytes. Therefore, it is possible to confirm the presence of adenovirus infection or astrogliosis induction through a reporter protein by linking the reporter protein to a downstream region of the GFAP promoter.

Additionally, it is possible to confirm the presence of adenovirus infection or astrogliosis by applying a different expression control sequence which enables the expression of genes in an astrocyte-specific manner to adenovirus instead of the GFAP promoter.

As used herein, the “vector” is a DNA capable of propagating by introducing a target DNA fragment into host bacteria or the like in a DNA recombination experiment, and is also referred to as a cloning vehicle. The vector may be a replication unit capable of bringing about replication of a DNA fragment bound thereto. A “replication unit” is any genetic unit (for example, plasmid, phage, cosmid, chromosome, or virus) that functions as a self-unit of DNA replication in vivo, that is, capable of replicating under its own control. In addition, a promoter for regulating the expression of a target fragment may be included in the vector.

In an embodiment, the vector of the present invention is a vector expressing hTauP301 L. When the vector is injected into a target animal, an AD animal model in which hTauP301 L is expressed can be prepared.

Specifically, the promoter included in the vector of the present invention may be EF1α or a GFAP promoter.

As the vector, for example, pWE15, M13, MBL3, MBL4, IXII, ASHII, APII, t10, t11, Charon4A, Charon21A, and the like may be used a phage vector or a cosmid vector, and a pBR system, a pUC system, a pBluescript II system, a pGEM system, a pTZ system, a pCL system, a pET system, and the like may be used as a plasmid vector. Specifically, an adeno-associated viral (AAV) vector, an adenovirus vector, a herpesvirus vector, a herpes simplex virus vector, a retrovirus vector, a lentivirus vector, and the like may be used as a virus vector. More specifically, the human mutant tau protein may be expressed using an adeno-associated viral vector.

Specifically, a transgenic animal may be constructed through the step of introducing a vector. As used herein, the term “transformation” refers to a method of altering the genetic trait of a cell by introducing a genetic material thereinto, and the transformation may be carried out by a conventional method in the art, and a method using a viral vector-based transfer method, a nonviral delivery technique using synthetic phospholipids, synthetic cationic polymers, and the like, electroporation in which a gene is introduced by applying a temporary electrical stimulation to the cell membrane, and the like may be used.

The vector of the present invention may include a fluorescent protein as a labeling material for confirming the expression of a target protein.

The fluorescent protein may be selected from the group consisting of, for example, green fluorescent protein (GFP), red fluorescent protein (RFP), blue fluorescent protein (BFP), yellow fluorescent protein (YFP), cyan fluorescent protein (CFP), enhanced fluorescent protein (EFP), and enhanced green fluorescent protein (EGFP).

In an embodiment of the present invention, a fluorescent protein was inserted into the vector to confirm the presence of hTauP301 L expression, and GFAP and a fluorescent protein were inserted into the vector to confirm the presence of adenovirus infection.

In the preparation method for an animal model of the present invention, the introduction of 1) and/or 2) may be, specifically, injecting the vector and/or adenovirus into the brain of an animal. The brain may include all of the cerebrum, cerebellum, hypothalamus, pituitary gland, amygdala, neonatal brain, substantia nigra, and the like without limitation, may be specifically the entorhinal cortex (EC), CA1, CA2, CA3, and CA4 of the lateral hippocampus, lateral ventricle (LV), subventricular zone (SVZ), subgranular zone (SGZ), and dentate gyrus (DG), and may be more specifically CA1 and dentate gyrus (DG).

As used herein, the term “injection” is not limited as long as the vector of the present invention and/or adenovirus can be introduced into an animal, and methods commonly used in the art may be used. For example, stereotaxic surgery may be used.

As used herein, the term “stereotaxic surgery” refers to a surgical operation in which a small target inside the body is located using a three-dimensional coordinate system, and some operations such as excision, biopsy, lesion, injection, stimulation, transplantation, and radiosurgery are performed.

In an embodiment of the present invention, the adeno-associated viral human mutant tau (AAV-hTau) vector and adenovirus (Adeno-GFAP-eGFP) were simultaneously injected into CA1 and DG of the hippocampus of a 5-month-old APP/PS1 transgenic mouse through stereotaxic surgery, and AD pathology was confirmed in the APP/PS1 transgenic mouse at the age of 8 months, which is earlier than before.

As used herein, the term “Alzheimer's disease”, also referred to as “AD, refers to the most common degenerative brain disease that causes dementia and was first reported by Dr. Alzheimer from Germany. It is known that as Alzheimer's disease progresses, the overall cognitive function including memory is gradually weakened. Extracellular amyloid plaques composed of Aβ peptides and intracellular NFTs composed of hyperphosphorylated tau protein are major neuropathological features of AD.

As used herein, the term “amyloid β (Aβ)” refers to peptides of 36 to 43 amino acids, which are critically involved in Alzheimer's disease as a main component of amyloid plaques found in the brains of patients with Alzheimer's disease. The peptides are derived from amyloid-beta precursor protein (APP), which is degraded by beta-secretase and gamma-secretase to produce Aβ.

As used herein, the “neurofibrillary tangles (NFTs)” are a disease caused by changes in nerve fibers. There is an argentaffin structure in the pyramidal cell body centered on the hippocampus in the brain of the general elderly people and throughout the cerebral cortex in the brain with Alzheimer's disease.

As used herein, the term “astrocyte” refers to one of the cells constituting the glia that supports the nervous tissue, and is most abundant in the CNS responsible for homeostasis of the brain, and contributes to protection in neuropathology by performing a number of functions, including providing nutrients to nervous tissue, maintaining extracellular ion balance, maintaining extracellular fluid homeostasis, regulating cerebral blood flow, repairing, and having a role in scarring.

As used herein, the term “astrogliosis” refers to an abnormal increase in the number of astrocytes due to destruction of surrounding neurons by trauma, infection, ischemia, stroke, autoimmune response, or neurodegenerative disease. Reactive astrogliosis, a hallmark of AD, activates the GFAP expression in astrocytes.

As used herein, the term “animal model” refers to a disease model that exhibits a disease through a disease model animal capable of showing the pathology of a specific disease, as in humans. As a representative example, models commonly used in relation to Alzheimer's disease include APP/PS1, 3×Tg, 5×Tg mouse models and the like.

The AD animal model of the present invention may be an animal having histological and behavioral features such as a decrease in the density of neurons, an increase in the reactivity of astrogliosis, an increase in the activity of MAO-B enzyme, cognitive impairment, and a decrease in memory, as well as disease pathological features such as an increase in tau pathology and acceleration of Aβ pathology at an earlier time than in the prior art.

In an embodiment of the present invention, immunohistochemistry and image quantification have been performed to examine whether GFAP, tau phosphorylation, Aβ, GABA, MAO-B, NFT, or NeuN activity have changed, and through this, it has been confirmed that GFAP has increased, tau phosphorylation has proceeded, Aβ number and size have increased, GABA has increased, MAO-B activity has increased, NFT has increased, and NeuN has decreased in APP/PS1-hTau/Adeno mice compared to the control group.

The animal model may include an animal other than a human, in which the onset of AD can be examined by inducing AD in the animal.

The animal includes, but is not limited to, for example, monkeys, dogs, cats, rabbits, guinea pigs, mice, cattle, sheep, pigs, and goats. In an embodiment of the present invention, mice were used, but the animal is not limited thereto.

The AD animal model of the present invention is an animal with increased tau pathology and accelerated Aβ pathology at 8 months old due to the introduction of the vector of the present invention and/or adenovirus, and may be an animal with markedly increased AD progression in histological and behavioral features.

In an embodiment of the present invention, the transgenic APP/PS1 mouse model exhibiting AD pathological features exhibited plaque pathology aggravated by overproduction of Aβ in the early stage of AD. Neuron loss and NFT pathology are not commonly observed in aged APP/PS1 mice even in the presence of Aβ. Most AD animal models do not simultaneously exhibit AD neurodegeneration, cognitive impairment, and neuropathological changes. However, the animal model prepared in the present invention is characterized by the occurrence of Aβ plaques at about 3 to 4 months old, and cognitive deficit and neuron loss at 8 months old.

Consequently, the mouse model according to the present invention can provide a technical method that can facilitate studies on AD target treatment strategies and tau pathology by providing behavioral and histopathological features.

Another aspect of the present invention provides an animal model prepared by way of the preparation method.

The “preparation method” and “animal model” are as described above.

As still another aspect, the present invention provides a screening method for a prophylactic or therapeutic agent for Alzheimer's disease, comprising (1) administering a test agent to an AD animal model; and 2) comparing the animal model of step (1) with a control animal model and selecting the test agent as a prophylactic or therapeutic agent for Alzheimer's disease when symptoms of Alzheimer's disease are improved.

The “AD”, “animal model”, and “Alzheimer's disease” are as described above.

As used herein, the “test agent” includes any substance, molecule, element, compound, entity, or combination thereof. The test agent includes, but is not limited to, for example, proteins, polypeptides, small organic molecules, polysaccharides, and polynucleotides. The test agent may also be a natural product, a synthetic compound, or a combination of two or more substances.

The test agent of the present invention includes, but is not limited to, all substances capable of inhibiting or eliminating amyloid-β plaque formation, reducing phosphorylation of tau protein, enhancing neurotransmission function, or preventing or delaying brain cell death.

As the method of administering the test agent, oral administration, intravenous administration, swabbing, subcutaneous administration, intradermal administration, topical administration, intranasal administration, intrapulmonary administration, rectal administration, or intraperitoneal administration may be used, and the administration method is not limited thereto, and may be appropriately selected depending on the symptoms of the experimental animal or the properties of the test agent. In addition, administration conditions such as the administration time or number of administrations of the test agent may be appropriately set to be optimal depending on the nature of the test agent, the purpose of testing and evaluation, and the like, and are not particularly limited. For example, for the timing of administration, a method of administering the test agent several times every 30 minutes immediately after construction of the model animal, then administering the test agent several times every one hour, and observing and evaluating the changes in symptoms each time is widely used. The number of administrations varies depending on the nature of the test agent, and may be one time or more or several times. The screening method for a test agent using the model animal of the present invention may be any method as long as it achieves the desired purpose.

As used herein, the term “control group” refers to an animal model that is not injected with the vector and/or adenovirus.

The substance obtained by this screening method acts as a leading compound in the subsequent AD prophylactic or therapeutic agent developing process, and it is possible to develop new prophylactic or therapeutic agents for brain diseases by modifying and optimizing the leading compound.

Consequently, the screening method according to the present invention can be usefully utilized to conduct physiological, molecular biological, and biochemical studies related to AD in animals including humans.

### Advantageous Effects

The preparation method for an animal model of Alzheimer's disease provided by the present invention may contribute to the development of the field of treatment technology for treating AD since the preparation method causes AD pathology as early as 8 months old and facilitates studies on AD target treatment strategies and tau pathology.

### BRIEF DESCRIPTION OF DRAWINGS

The patent or application file contains a least one drawing executed in color. Copies of this patent or patent application publication with color drawing(s) will be provided by the Office upon request and payment of the necessary fee.

FIG. 1 illustrates accelerated tau pathology with severe astrogliosis in an APP/PS1-hTau/Adeno AD mouse model; (A) a schematic diagram and representative fluorescence image showing the injection site of AAVDJ-EF1α-hTauP301L-GFP (AAV-hTau; hTau) and Adeno-GFAP-GFP (Adeno-GFAP; Adeno) (scale: 1 mm); (B) experimental timeline; (C) immunohistochemical images of S199 and GFAP in adult brain sections from 8-month-old WT-CTL (n=4), APP/PS1-CTL (n=4), APP/PS1-hTau (n=5) and APP/PS1-hTau/Adeno (n=6) mice, phospho-tau (red; using S199) and astrocytes (blue; using GFAP) are immunolabeled in brain slices, and each point is the mean value measured in 3 to 5 sections per mouse (scale: 40 μm); (D) phospho-tau-5199 intensity increases in APP/PS1-hTau/Adeno mouse hippocampal slices; (E) GFAP immunoreactive astrocytes are activated in the CA1 region, and activation of the APP/PS1-hTau group is similar to that of the APP-CTL group, but the activity of the APP/PS1-hTau/Adeno group significantly increases compared to other groups, (p<0.05 (*), p<0.01 (**) and p<0.001 (***)); and (F) 3D images are reconstructed from confocal images using IMARIS software;

FIG. 2 illustrates enhanced amyloid pathology following induction of human mutant tau protein overexpression and reactive astrogliosis; (A) representative immunofluorescence images of human and mouse Aβ (yellow) staining in hippocampal CA1 sections from 8-month-old mice injected with AAV-hTau and Adeno-GFAP (grey) (scale: 40 μm); (B) representative images of amyloid plaques detected with thioflavin-S (thio-S) from WT-CTL (n=4), APP/PS1-CTL (n=8), APP/PS1-hTau (n=10) and APP/PS1-hTau/Adeno (n=6) mice, Aβ is absent in WT mice injected with the control virus vector, but is progressively deposited in the somatosensory cortex (S1) and piriform cortex (Pir) of APP/PS1 mice injected with human tau and adenovirus at 3 mpi (scale: 1 mm); and (C) Aβ plaque quantification (p<0.05 (*) and p<0.01 (**)), and each point is the average for several sections from a single animal;

FIG. 3 illustrates an increase in astrocyte-derived GABA release by increased reactive astrocytes and MAO-B activity in an APP/PS1-hTau/Adeno mouse model; (A) immunostaining and quantification of GABA in the CA3 molecular layer, and arrowheads denote GABA-positive astrocytes (both male and female are 8 months old, scale: 10 μm); (B) GFAP/GABA double positive cells are quantified in hippocampal CA3 of WT-CTL (n=4), APP/PS1-CTL (n=4), APP/PS1-hTau (n=4) and APP/PS1-hTau/Adeno (n=4), each point is the average for four sections from a single animal, and the mean intensity of GABA in GFAP-positive regions (p<0.05 (*), p<0.01 (**); (C) response diagram in MAO-B activity assay in hippocampus (wildtype n=2; other groups n=3; left or right hippocampus is analyzed; 8 months old); (D) representative images of enzyme activity levels in the MAO-B assay, and excitation at 560 nm±10 nm, fluorescence detection at 590 nm±10 nm, and measurement of fluorescence using a fluorescence microplate reader; (E) MAO-B produces GABA in reactive astrocytes, and MAO-B enzyme activity is measured in the hippocampus of WT-CTL, WT-hTau, APP/PS1-CTL, APP/PS1-hTau, and APP/PS1-hTau/Adeno samples by Amplex red monoamine oxidase assay (***p<0.001, *p<0.05 vs. WT-CTL; ###p<0.001, ##p<0.01 vs. WT-hTau);

FIG. 4 illustrates the production and accumulation of aberrant tau protein form in the APP/PS1-hTau/Adeno mouse brain; (A) representative photomicrographs by ABC/DAB staining of NFTs in brain sections of 8-month-old APP/PS1-hTau/Adeno mice with tauopathy, and the NFT pathological tau species is detected by human NFT with a conformation-dependent anti-tau monoclonal antibody (scale: 500 μm); (B, C) quantification of NFT intensity in WT-hTau/Adeno (n=4), APP/PS1-hTau (n=5) and APP/PS1-hTau/Adeno (n=6) brains compared to WT-CTL (n=6) (p=0.0169, (B), p=0.0141, 0.0462, (C)), induction of overexpression of P301 LhTau and reactive astrogliosis increases NFT+ cells in APP/PS1 mice with tau pathology, and an increased number of NFT+ cells is observed in hippocampal CA1 pyramidal cells (CA1 Py) (B) and dentate gyrus cells (DGGC) (C) (p<0.01 (**), p<0.001 (***)); (D) expression of P301 L hTau in CA1 and DG induces tau phosphorylation and accumulation, representative confocal images of AT8 and NFT (scale: 40 μm), and each point is the average for several cells from a single animal; (E) quantification of p-Tau intensity in GFAP-positive regions, and the phosphorylation level of tau significantly increases in the fibers projecting to CA1 and DG in APP/PS1-hTau/Adeno mouse brain tissue in three months after injection of AAV-hTau and adenovirus; and (F) quantification of NFT intensity of hippocampus in WT-hTau, APP/PS1-CTL, and APP/PS1-hTau/Adeno brain sections;

FIG. 5 illustrates neuronal cell death in the CA1 region of the hippocampus by overexpression of tau protein and reactive astrogliosis; (A) the number of neurons significantly decreases in the CA1 pyramidal layer of APP/PS1-hTau/Adeno mice (scale: 40 μm); (B) quantification of the number of NeuN-positive cells results in a significant decrease in P301 L human mutant tau overexpression and reactive astrogliosis in APP/PS1-hTau/Adeno mice, and each point is the average for several sections from a single animal (p<0.05 (*));

FIG. 6 illustrates that hTau overexpression and reactive astrogliosis do not affect anxiety-related behaviors in open field test results; (A) heat-map showing tracks of mice from different groups in the OFT (red=more time, blue=less time); (B) total distance; (C) central distance; and (D) time in center, anxiety is not reduced, and search activity is not reduced in aged APP/PS1-hTau/Adeno mice, WT-CTL (n=5), APP/PS1-CTL (n=3), APP/PS1-hTau (n=3), and APP/PS1-hTau/Adeno (n=6);

FIG. 7 illustrates memory and cognitive impairment in the APP/PS1-hTau/Adeno mouse model; (A) schematic representation of sample and test steps in NORT; (B) heat-map showing mouse tracks in NORT (red=more time, blue=less time); (C) comparison of discrimination index (DI) of WT littermates of the same age as APP/PS1 mice injected with CTL, AAV-hTau or Adeno-GFAP and tested in NORT, and the D2 recognition index is used to control the total search time: D2=new/(new+familiar), dashed lines denote opportunity runs (0.5) (p<0.05 (*), p<0.01 (**) and p<0.001 (***)); (D) Y-maze test; (E) heat-map showing overall activity and Y maze top view of control mice (left) and APP/PS1-hTau/Adeno mice (right); (F) Y maze test shows that spontaneous alternation behavior is impaired by induction of P301 L hTau overexpression and reactive astrogliosis, and APP/PS1-hTau/Adeno mice (8 months old) exhibit significantly reduced spontaneous alternation behavior compared to littermate WT mice (p<0.05 (*) and p<0.01 (**)); (G, H) APP/PS1-hTau/Adeno mice exhibit impaired fear memory in the passive avoidance test, the passive avoidance test of WT and APP/PS1 injected after three months, mice received double training times with 0.4-mA electric shock for 2 seconds, the test is performed immediately 24 hours after the training time (**P<0.01), compared with WT, WT-CTL (n=6); WT-hTau (n=5); WT-hTau/Adeno (n=5); APP/PS1-CTL (n=7); APP/PS1-hTau (n=8); and APP/PS1-hTau/Adeno (n=7); and

FIG. 8 is a schematic diagram illustrating the progression of reactive astrogliosis and overexpression of human mutant tau protein (P301 L hTau).

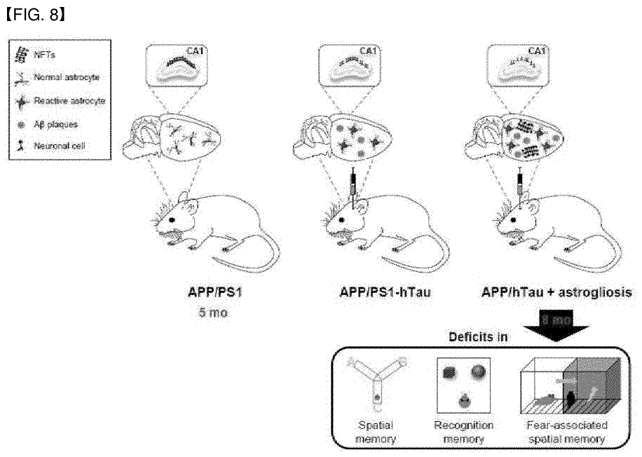

### DETAILED DESCRIPTION OF THE INVENTION

Hereinafter, the present invention will be described in more detail with reference Examples to help the understanding of the present invention. However, the following Examples are merely illustrative of the contents of the present invention, and the scope of the present invention is not limited to the following Examples. Examples of the present invention are provided to more completely explain the present invention to those of ordinary skill in the art.

### Example 1. Construction of Animal Model of Alzheimer's Disease

### Example 1-1. Preparation of Animal Model

A 5-month-old APPswe/PSEN1dE9 (APP/PS1) hemizygote from the C57BL/6; C3H genetic background (Jackson Laboratory, #004462) used in all experiments was used. As a control group (wild-type, WT), non-transgenic mice of a similar age group born in litter were used. The transgenic mice and the control group include both females and males, had free access to food and water, and were maintained on a 12-hour light-dark cycle. The animal care and handling entirely conform to KIST's Institutional Animal Care and Use Committee.

### Example 1-2. Construction of Animal Model Expressing Alzheimer's Disease

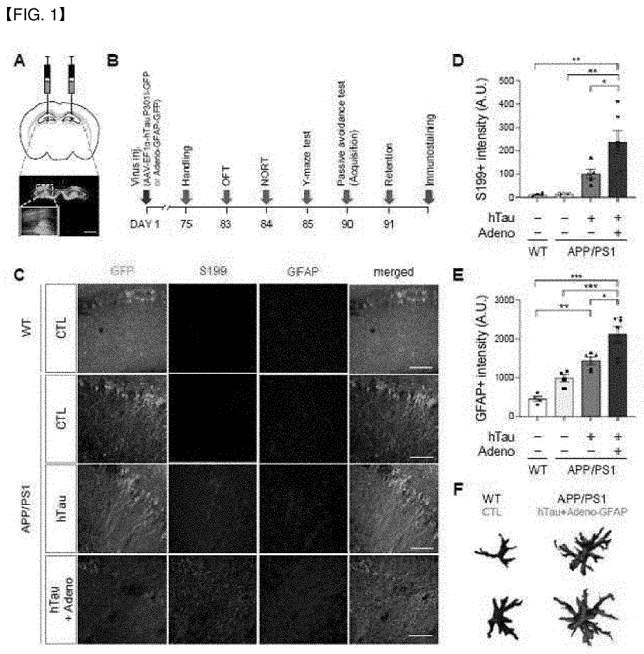

In order to show the tau pathology and tau accumulation pattern in AD caused by tau overexpression, AAVDJ-EF1α-hP301L-GFP (AAV-hTau; hTau) and Adeno-GFAP-GFP (Adeno-GFAP; Adeno) were constructed. First, AAVDJ-EF1α-hP301L-GFP was constructed by introducing an EF1α promoter sequence, a sequence encoding a mutant protein introducing a P301 L mutation in the known Tau protein sequence (Gene ID: 4137), and GFP to an adeno-associated human mutant vector. The Adeno-GFAP-GFP was constructed by introducing a GFAP promoter (GLIA 56:481-493 (2008)) and a GFP protein to adenovirus. Thereafter, AVDJ-EF1α-hP301L-GFP and Adeno-GFAP-GFP were injected into CA1 and DG of bilateral hippocampus of APP/PS1 mice or their littermates (5 months old). As a control group, an AAVDJ-EF1α-GFP (AAV-CTL; CTL) vector of the same volume was constructed and used for injection (FIG. 1A).

APP/PS1-hTau mice were constructed by injecting hTau into APP/PS1, and APP/PS1-hTau/Adeno mice were constructed by injecting both hTau and Adeno into APP/PS1 mice.

In this experiment, a control group WT-CTL in which any mouse model was not treated, a control group WT-hTau in which hTau was injected into any mouse model, a control group WT-hTau/Adeno in which hTau and Adeno were injected into any mouse model, a control group APP/PS1-CTL in which the APP/PS1 model was not treated, a control group APP/PS1-hTau in which hTau was injected into the APP/PS1 model, and APP/PS1-hTau/Adeno in which hTau and Adeno were injected into the APP/PS1 model were prepared.

### Examples 1-3. Stereotaxic Surgery

All AAV vector plasmids were prepared at the virus facility of KIST (Korea Institute of Science and Technology, Seoul). In order to express human P301L mutant tau (hTau) in neurons, 5-month-old APP/PS1+/+ or APP/PS1−/−mice were briefly anesthetized with isoflurane and placed in a stereotaxic frame (Kopf Instruments). After craniotomy, holes were drilled on both sides using a drill handpiece. Virus suspensions were microinjected using a 25 μL Hamilton syringe and a 33-gauge blunt needle connected to an automated micro-syringe pump (KD Scientific, Holliston, Mass., USA). AAV_{DJ}-EF1α-GFP (CTL), AAV_{DJ}-EF1α-hTauP301 L-GFP (AAV-hTau; P301LhTau, hTau) or Adeno-GFAP-GFP (Adeno-GFAP; Adeno) was injected bilaterally into CA1 and dentate gyrus of the hippocampus. The coordinates are: AP=−1.96; ML=±1.5; DV=−1.6 to 1.7 dura mater surface. Virus stock was injected at a rate of 2 μL/min. Prior to withdrawal, the needle was held in place for an additional 5 minutes to prevent leakage of the injected solution. After injection, the skin was sutured and the mice were kept warm on the heating pad to allow them to fully recover before being sent to the home cage.

### Example 2. Verification of APP/PS1-hTau/Adeno Mouse

### Example 2-1. Immunohistochemistry

Mice were anesthetized with 2% avertine (avertin, 20 μg/g, i.p.) (Sigma-Aldrich; St. Louis, Mo.) in 13 weeks after virus injection. Anesthetized mice were transcardially perfused with phosphate-buffered saline (PBS, pH 7.4) and then perfused with PBS containing 4% paraformaldehyde. After perfusion, the tissue was immediately removed, post-fixed in the same fixative for 12 hours, and then dehydrated in 30% sugar until it subsided. Brains were cut coronal in a 30 μm-thick frozen microtome (Thermo Scientific, Waltham, Mass., USA).

To block non-specific binding sites, the sections were incubated in 0.1 M PBS containing 0.3% Triton X497 100 (Sigma), 2% goat serum (ab7481, Abcam), and 2% donkey serum (GTX27475, Genetex) for 2 hours at room temperature. The primary antibody prepared in the blocking buffer was overnight at 4° C. After washing three times with 0.1 M PBS at room temperature for 5 minutes, the brain slices were incubated with appropriate secondary antibodies from Jackson Laboratory at room temperature for 1.5 hours. The sections were washed three times with 0.1 M PBS, and fluorescent inclusion bodies were fixed in 0.1 M PBS. A series of fluorescence images were taken with an A1 Nikon confocal microscope. Z-stack projections were prepared from a series of images in 3 μm steps. Changes in brightness or contrast were applied equally to the entire image set. 3D reconstruction of all Z-stacks was confirmed using IMARIS software (Bitplane).

The brain sections were stained using chicken anti-GFAP (1:500, ab5541, Millipore), mouse anti-phosphorylated tau AT8 (pSer202 and pThr205; 1:200, MN1020, Thermo Fisher Scientific), rabbit anti-phosphorylated human tau S199 (pSer199; ab81268, Abcam), rabbit anti-neurofibrillary tangle (1:200, ab1518, Millipore), and rabbit anti-beta-amyloid (1:500, ab2539, Abcam), and guinea pig anti-NeuN, ABN90P, Millipore).

For neurofibrillary tangle (NFT) staining, the sections were incubated in PBS containing 3% hydrogen peroxide at room temperature for 10 minutes, and washed with PBS three times for 15 minutes. To block non-specific binding sites, the sections were incubated in PBS containing 3% normal goat serum (Life technologies) and 3% Triton X-100 at room temperature for 1 hour. The sections were incubated overnight at 4° C. together with a primary antibody against NFT (1:400, ab1518, Milipore). After washing with PBS three times, the sections were applied to the secondary antibody of biotinylated goat anti-rabbit (Vector Laboratories) diluted in PBS to 1/200 at room temperature for 2 hours. Vectastain ABC Elite kit (PK6101, Vector Laboratories) was used for peroxidase detection according to the manufacturer's instructions. Then, the sections were washed and expressed with hydrogen peroxide in DAB (Sigma Aldrich) and TBS. The DAB-stained sections were fixed on microscope slides, dried in the air, and covered with a coverslip on top. Images were obtained using Olympus IX50 microscope at 2× or 4× magnification. For quantitative analysis, ImageJ software was used to measure the average optical density.

Immunohistochemistry was performed in three months after the injection of the vector and adenovirus, and it was confirmed that APP/PS1-hTau/Adeno mice showed more consistent and abundant expression of plaques and tau tangles than APP/PS1 mice (FIG. 1B).

### Experimental Example 2-2: Image Quantification

IMARIS software (Bitplane) was used for quantitative analysis by a confocal microscope. Regions of interest (ROIs) were determined as 3D structures of GFP-positive regions using “surface objects” created using a 0.312 μm diameter. Smaller and adjacent neurons were separated into multiple entities using the “split touching objects” function of estimated 6 μm diameter. Thus, large puncta were potentially dissociated into several individual neurons. Then, the average intensity values of pixels of different wavelengths (405 nm, 594 nm, and 647 nm) were measured by masking them with GFP-positive cells. After behavioral experiments, at least four mice per group were sacrificed, and 2 to 3 slices per mouse were stained and quantitatively analyzed.

### Example 2-3. Mouse Tau Phosphorylation by Mutant Human Tau and Reactive Astrogliosis

APP/PS1 transgenic mice were injected with AAV-hTau and adenovirus (APP/PS1-hTau/Adeno). Then, immunohistochemistry of the specific antibody of p-Tau (S199) was performed on APP/PS1 mice and littermates. p-Tau, a main constituent of paired helical filaments (PHFs) in AD, was detected in the brain tissue of APP/PS1 (5-month-old) mice at 3 mpi, unlike WT mice. As expected, APP/PS1-hTau/Adeno mice showed excessive p-Tau expression in hippocampal CA1 (FIG. 10). p-Tau accumulation was significantly increased by almost 2-fold in APP/PS1-hTau/Adeno mice (237.10±50.33) compared to APP/PS1-hTau mice (101.55±19.86, p<0.01) (FIG. 1D). Compared to APP/PS1-CTL mice (13.19±2.46, ns), CA1 of 8-month-old APP/PS1-hTau mice (19.67±5.01) showed a tendency to increase, but did not reach statistical significance (FIG. 1C). This means that mutant human tau and reactive astrogliosis increase tau phosphorylation in mice.

GFAP levels were significantly increased in the CA1 region of the hippocampus in APP/PS1-hTau/Adeno mice (2104.75±218.52) compared to WT-CTL (452.25±63.98, p<0.0001), APP/PS1-CTL (986.34±100.38, p<0.001), or APP/PS1-hTau (1433.58±101.21, p<0.05) mice (FIGS. 1E and 1F). GFAP was significantly increased in APP/PS1-hTau mice compared to WT mice (p<0.005). However, APP/PS1-CTL mice (p=0.1749, ns) showed a higher amount of GFAP than littermates, but the difference was not significant (FIG. 1E). These dramatic differences indicate redistribution of the intermediate filament network by reactive astrogliosis and overexpression of the tau protein in APP/PS1 mice.

In other words, these results show that overexpression of mutant human tau and reactive astrocytes can induce a certain level of phosphorylation of tau in the mouse hippocampus. However, since tau in the P301 L mutant form tends to accumulate over time, tau pathology and misfolding of related tau protein were further analyzed.

### Example 2-4. Acceleration of Amyloid Pathology by Overexpression of Human Mutant Tau and Reactive Astrogliosis in APP/PS1 Mice

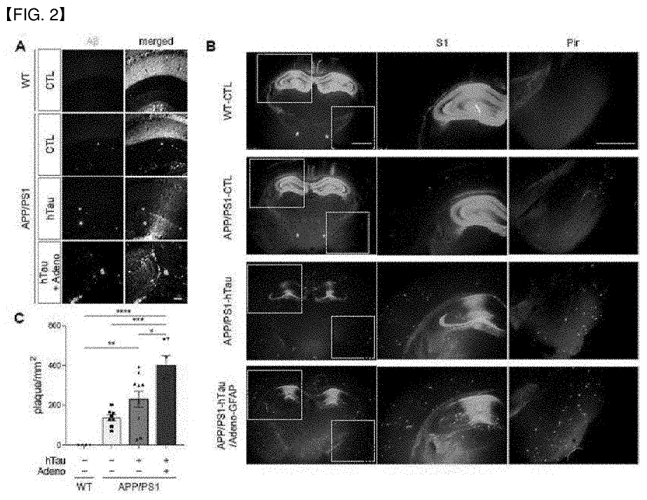

In APP/PS1-hTau/Adeno mice, the number and size of Aβ were increased (FIG. 2A). Thioflavin-S staining was performed on 8-month-old APP/PS1 mice to make AD mouse brain sections. Aβ accumulation was more abundant in the piriform cortex and somatosensory cortex, while slightly extending into the hippocampus. Aβ accumulation was observed in APP/PS1 mice, but not in WT (FIG. 2B). The number of thioflavin-S+ plaques in APP/PS1-hTau/Adeno mice (401.44±49.12) was significantly increased compared to APP/PS1-CTL (136.64±14.82) and APP/PS1-hTau (230.16±40.75) mice, and this indicates that Aβ accumulation was accelerated by the overexpression of human mutant tau and severe reactive astrogliosis (FIG. 2C).

### Example 2-5. Increased Astrocyte-Derived GABA Activity in APP/PS1-hTau/Adeno AD Mouse Model

Since GABA levels are abnormally increased in patients with Alzheimer's disease and this increase in GABA is caused by MAO-B, it was verified whether this pathology also appeared in the mouse model constructed in the present invention.

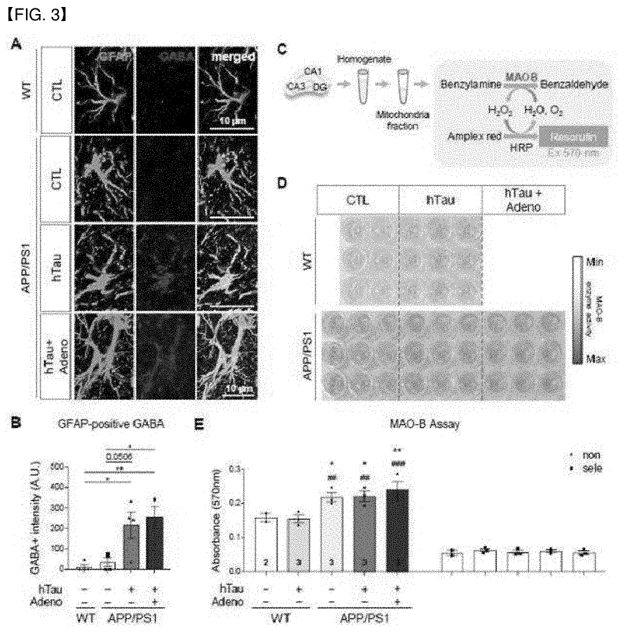

In order to investigate the aberrant increase in GABA levels from AD mouse reactive astrogliosis, co-immunostaining was performed on hippocampal slices with antibodies against GABA and GFAP. It was found that normal astrocytes showed a minimal immune response to GABA in 8-month-old WT mice and APP/PS1-CTL mice. Conversely, reactive astrocytes were strongly immunoreactive in APP/PS1-hTau and APP/PS1-hTau/Adeno mice (FIG. 3A). Astrocyte GABA levels in the molecular layer of CA3 observed in APP/PS1-hTau and APP/PS1-hTau/Adeno mice (p<0.05 and p<0.001, respectively) were significantly increased in a manner similar to the increase in immune response to GFAP (FIG. 3B). GABA intensity of GFAP-positive astrocytes in APP/PS1-hTau/Adeno mice was significantly increased compared to APP/PS1-CTL mice (p<0.05). Conversely, GABA intensity was increased in APP/PS1-hTau mice, but there was no significant difference (FIG. 3B).

### Example 2-6. Increased MAO-B Activity in APP/PS1-hTau/Adeno AD Mouse Model

Animals were anesthetized with avertin through intraperitoneal injection, and perfused transcardially with saline. The brain tissue was removed from six mouse groups. Other regions, including hippocampus and cortex, were separately dissected at a low temperature and stored at −80° C. prior to analysis. Fresh tissue from each mouse was homogenized in lysis buffer (250 mM sucrose, 2 mM HEPES (pH 7.4), 0.1 mM EGTA), and centrifuged at 570 g for 10 minutes to remove large debris. The supernatant was centrifuged at 14,290 g for 10 minutes to obtain a mitochondrial-rich fraction. The pellet was resuspended in phosphate buffer, and MAO-B activity was measured using 20 μg in each well. The enzymatic activity of MAO-B was measured using Amplex Red Monoamine Oxidase Assay Kit (Molecular Probes) according to the manufacturer's instructions. MAO-B was reacted with or without addition of selegiline, a MAO-B inhibitor at 37° C. for 30 minutes, and then reacted with benzylamine, a MAO-B substrate. After 2 hours of enzymatic reaction, the degree of generation of hydrogen peroxide by MAO-B activity was measured by the color change of Amplex red reagent. The color change was quantified by measuring the absorbance at 570 nm using Infinite M200 PRO microplate reader (TECAN).

In order to examine whether monoamine oxidase-B (MAO-B) activity increased in the APP/PS1-hTau/Adeno AD mouse model, fluorescence analysis of MAO-B was performed. A mitochondrial-rich fraction was prepared from tissue homogenates of half of the isolated hippocampus (FIG. 3C). Compared to WT astrocytes, MAO-B expression in reactive astrocytes of APP/PS1 mice was significantly increased (FIG. 3D), and MAO-B expression was higher in APP/PS1-hTau/Adeno mice. MAO-B activity was significantly increased to 38.14%, 39.48%, and 52.34% in APP/PS1-CTL mice, APP/PS1-hTau mice, and APP/PS1-hTau/Adeno mice, respectively (FIG. 3E). Moreover, enhanced MAO-B activity was recovered in all groups treated with selegiline, a selective and irreversible inhibitor of MAO-B.

### Example 2-7. Facilitation of Tau Deposition by Synergistic Effect of Human P301L Tau and Reactive Astrogliosis in APP/PS1 Mice

Abnormal tau protein folding may precede the formation of PHF and neurofibrillary tangles (NFT), one of the neuropathological features of AD. It shows that NFTs contribute to an increase in the incidence of neurodegenerative diseases as AD progresses.

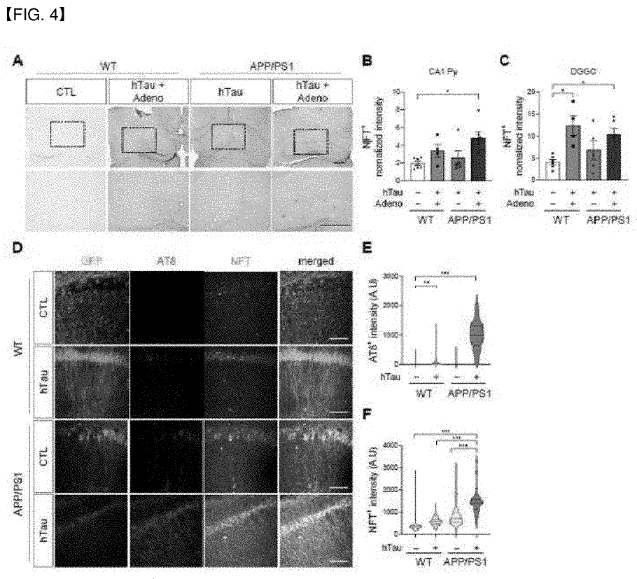

The presence of NFT was analyzed in the brains of four mouse groups (WT-CTL; WT-hTau/Adeno; APP/PS1-CTL; and APP/PS1-hTau/Adeno) immunostained with an antibody against human NFT. A strong signal appeared exclusively in the projection fibers in the hippocampal pyramidal layer of CA1 and the intermediate molecular layer (mml) of DG (FIG. 4A). Except for the CA1 pyramidal layer and the neural body of DG, no other parts of the hippocampus showed NFT-positive signals, and this indicates that the exogenous mutant tau protein did not diffuse into other brain regions but was restricted in CA1 and DG. All of the APP/PS1-hTau/Adeno mice showed significantly increased NFT levels in CA1 (4.83±0.70, p<0.05) and DG (10.23±1.49, p<0.05) of the hippocampus compared to WT mice (CA1, 1.968±0.25; DG, 4.09±0.59) (FIGS. 4B and 4C). Although APP/PS1-hTau mice (CA1, 2.55±0.82; DG, 6.767±2.17) showed a higher tendency to aggregate NFT tau compared to WT-CTL mice, the difference was not significant.

Immunohistochemistry double staining was performed with an antibody against the phosphorylated region of tau protein (AT-8) and NFT. It was confirmed that GFP and AT-8 were co-located in the neuron cell bodies of CA1 and DG and the projection fibers of the DG region (FIG. 4D). Unlike the WT-CTL group, a gradual accumulation of tau as a pathological form occurred in hippocampal CA1 of the APP/PS1-hTau group (FIGS. 4D and 4E). The level of tau accumulation in the group in which the AAV-hTau vector was co-injected into APP/PS1 mice increased by 4-fold and 1.6-fold, respectively, compared to the WT-CTL and APP/PS1-CTL groups injected three months prior (FIG. 4F). This means that the P301 L pathogenic tau mutation and reactive astrogliosis can increase tau phosphorylation and that its aggregation and modification can result in deposition in the hippocampus of mice.

### Example 2-8. Facilitation of Neurodegeneration in CA1 Region of Hippocampus in APP/PS1-hTau/Adeno Mice

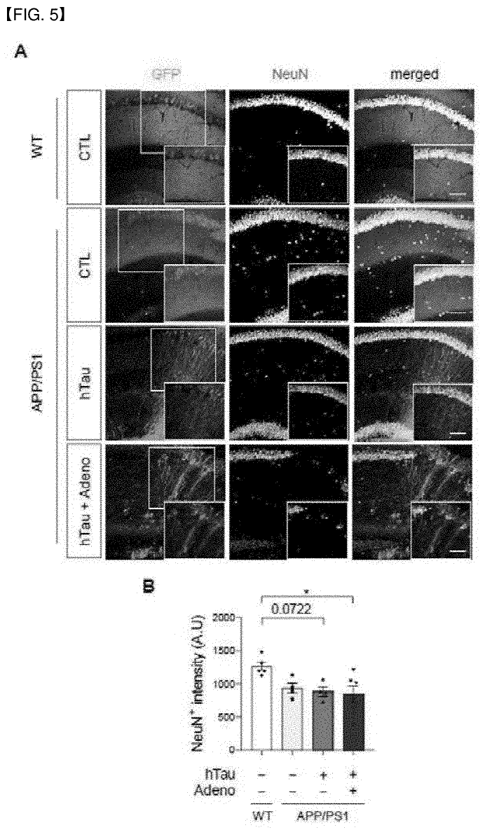

In order to evaluate whether induction of human mutant tau and reactive astrogliosis contributes to potential neuron loss, NeuN was used as a specific neuronal marker. Analysis of NeuN signals located in CA1 showed significant differences between groups (FIG. 5A). As a control, WT-CTL mice showed a uniform distribution of NeuN positive signals in somatic cells of hippocampal CA1 pyramidal neurons. There was no significant difference in the number of neurons between WT-CTL mice (1265.56±62.17) and APP/PS1-CTL (939.04±74.98) and APP/PS1-hTau (884.33±71.42) mice (FIG. 5B). Compared to the WT control group of the same age, the number of NeuN-positive cells in APP/PS1-hTau mice tended to decrease, but there was no significant difference. However, neuron loss in the CA1 pyramidal layer of APP/PS1-hTau/Adeno mice (841.68±124.70, p<0.05) was significantly reduced compared to the 8-month-old control group (FIG. 5B). Through this, it has been confirmed that the gradual neuronal cell death is shown at 8 months old, that is, in three months after simultaneous injection of AAV-hTau and Adeno-GFAP, and a pathology similar to human AD appears in the present invention, whereas neuron loss conventionally occurs at 17 months old.

### Example 3. Behavior Analyses

In order to investigate whether the expression of human mutant tau protein and reactive astrogliosis induce learning and memory impairment in AD mice, four types of behavioral experiments to evaluate hippocampal-dependent memory and cognition were performed on six groups (WT-CTL; WT-hTau, WT-hTau/Adeno, APP/PS1-CTL, APP/PS1-hTau, and APP/PS1-hTau/Adeno).

### Example 3-1. Anxiety and Mobility in APP/PS1-hTau/Adeno AD Mouse Model (OF Test)

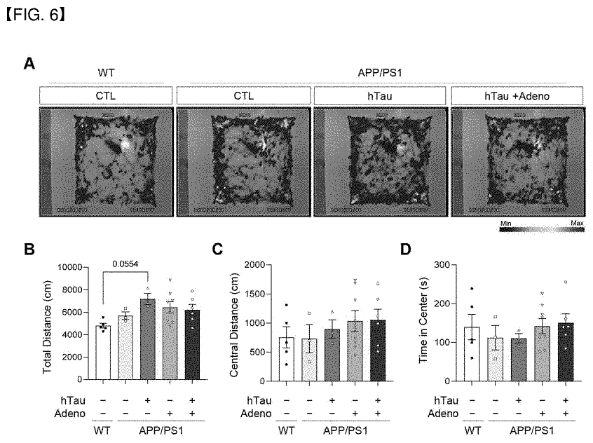

In order to examine the effect of hTau overexpression and astrogliosis on mouse behavior, an open field (OF) test was performed to establish anxiety and basic motor function of APP/PS1-hTau/Adeno mice injected three months prior and WT-CTL mice (FIG. 6A). The total distance moved by the mouse represents the degree of movement and activity. APP/PS1-hTau, APP/PS1/Adeno, and APP/PS1-hTau/Adeno mice all tended to be more active than the WT control group in the OF test (FIG. 6B). The effect of mutant tau overexpression significantly increased activity (p=0.0127). In order to evaluate the anxiety-related behaviors of APP/PS1-hTau/Adeno mice or WT-CTL mice, the time spent in the peripheral and central regions of the open field arena was measured. In the central region of the field, no significant difference was observed in the required time and movement distance of each mouse (FIGS. 6B and 6C).

Based on this, it was confirmed that anxiety and motor ability did not affect the cognitive ability—related behavior analysis of Example 3-2.

### Example 3-2. Hippocampal-Dependent Memory and Cognitive Impairment by P301L Human Mutant Tau Overexpression and Severe Reactive Astrogliosis (NOR, Y-Maze, PA Test)

In order to evaluate the cognitive impairment of APP/PS1-hTau/Adeno, the behavioral analysis of four mouse groups (WT-CTL, APP/PS1-CTL, APP/PS1-hTau, APP/PS1-hTau/Adeno) in which the vector prepared in Example 1 and adenovirus were injected into WT and APP/PS1 mice 13 weeks prior.

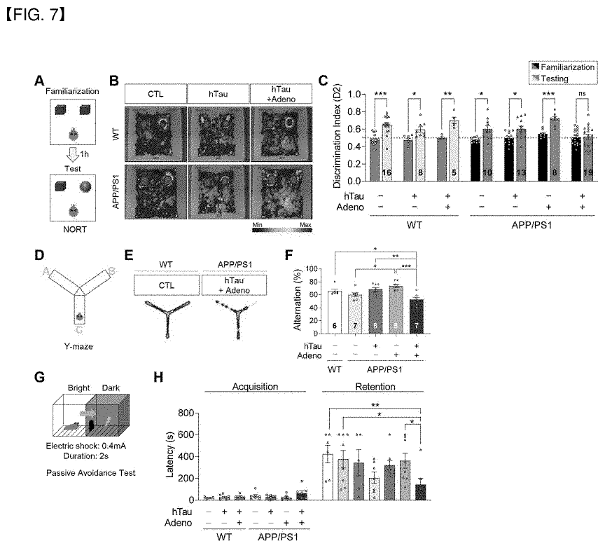

First, in the novel object recognition (NOR) test, all groups of 8-month-old mice were tested for hippocampal-dependent function impairment (FIG. 7A). During the training phase, the mice were exposed to two identical objects for 30 minutes. In the familiarization phase of the mouse test, the object interaction rate in all six groups was between 47.5% and 50.7%. One hour after the training phase, WT mice (increased by 17.5% compared to the discrimination index during the training course, p<0.0001) and APP/PS1 mice overexpressing human mutant tau (increased by 10.6%, p=0.0207) or APP/PS1 mice not expressing human mutant tau ng (increased by 12.6%, p=0.0135) APP/PS1 mice were able to discriminate between familiar and new objects (FIGS. 7B and 7C). In APP/PS1-hTau/Adeno mice, there was no significant difference in the interaction between the sample and the new object, and this indicates that the recognition ability to discriminate a new object is impaired (p>0.9999) (FIGS. 7B and 7C).

Spatial memory was assessed through a Y-maze test. In the three-armed Y-shaped maze (FIG. 7D), mice were allowed to navigate freely, and the order of entry was recorded to determine the number of consecutive visits on the three different arms. The analyzed percent change reflects hippocampal function of the subject mouse, and a higher percent change indicates better spatial memory. The total number of arm entries did not differ significantly between groups, but it was confirmed that the rate of spontaneous change was significantly reduced in APP/PS1-hTau/Adeno mice (52.62±3.67) compared to WT-CTL mice (65.85±2.89) or APP/PS1-hTau mice (68.39±3.22) (FIGS. 7E and 7F). This indicates that the hippocampal-dependent recognition function of APP/PS1-hTau/Adeno mice is impaired.

In order to measure the effect of P301 L hTau and reactive astrogliosis on hippocampus-dependent fear memory, a passive avoidance (PA) test to evaluate fear-related spatial memory was performed (FIG. 7G). This test allows rodents to lose their affinity for dark rooms and remain their affinity for bright rooms. During the acquisition phase, all animals took an average of 35 seconds to enter the darkroom, and this indicates no baseline differences in anxiety between groups in this behavioral analysis. After 24 hours, the delay rate of avoiding the dark room was measured to evaluate how well the mouse remembered the shock. The latency of the APP/PS1-hTau/Adeno mouse group for the dark room entry of the passive avoidance device was significantly increased (p<0.01) compared to the WT group during the retention period (FIG. 7H). Conversely, APP/PS1-hTau showed no significant difference compared to the WT control group, and this excluded the possibility that human tau itself might affect fear memory. The APP/PS1-CTL mice also did show any significant difference from the WT mice (p<0.06, FIG. 7H), but there was a tendency to decrease.

Based on the above description, it will be understood by those skilled in the art that the present invention may be implemented in a different specific form without changing the technical spirit or essential characteristics thereof. Therefore, it should be understood that the embodiments are not limitative but illustrative in all aspects. The scope of the present invention is defined by the appended claims rather than by the description preceding them, and therefore all changes or modifications derived from the meaning and scope of the claims and their equivalents should be construed as being included in the scope of the present invention.

1. A preparation method for an animal model of Alzheimer's disease (AD), comprising injecting:
1) a human mutant tau protein expression vector; and
2) adenovirus into an animal other than a human.

2. The preparation method according to claim 1, wherein the human mutant tau protein of 1) has a P301L mutation.

3. The preparation method according to claim 1, wherein the vector of 1) is an adeno-associated viral (AAV) vector.

4. The preparation method according to claim 1, wherein the adenovirus of 2) comprises a GFAP promoter and a reporter protein as a constitution for confirming whether or not astrogliosis is induced.

5. The preparation method according to claim 1, wherein the vector of 1) and the adenovirus of 2) are to be injected into CA1 and dentate gyrus (DG) of hippocampus of an animal.

6. The preparation method according to claim 5, wherein the injection is performed through stereotaxic surgery.

7. The preparation method according to claim 1, wherein the animal model is a mouse.

8. The preparation method according to claim 7, wherein the mouse is an APP/PS1 mouse.

9. The preparation method according to claim 1, wherein the preparation method is a preparation method for an animal model prepared by inducing overexpression of human mutant tau protein and astrogliosis.

10. The preparation method according to claim 1, wherein the animal model has one or more symptoms of NFT pathology, neuron loss, Aβ pathology, and acceleration of astrogliosis at 8 months old.

11. An animal model prepared by way of the preparation method according to claim 1.

12. A screening method for a prophylactic or therapeutic agent for Alzheimer's disease, comprising:
1) administering a test agent to the animal model of claim 11; and
2) comparing the animal model of step 1) with a control animal model and selecting the test agent as a prophylactic or therapeutic agent for Alzheimer's disease when symptoms of Alzheimer's disease are improved.
